(19) United States
(12) Patent Application Publication
(10) Pub. No.: US 2023/0089985 A1
(43) Pub. Date: Mar. 23, 2023

(54) FLEXIBLE SANITIZING APPARATUS

(71) Applicants: Michelle Zhiya Li, Watkinsville, GA (US); Tianjie Lu, Watkinsville, GA (US); Barnabas Huamu Li, Watkinsville, GA (US)

(72) Inventors: Michelle Zhiya Li, Watkinsville, GA (US); Tianjie Lu, Watkinsville, GA (US); Barnabas Huamu Li, Watkinsville, GA (US)

(21) Appl. No.: 17/946,867

(22) Filed: Sep. 16, 2022

Related U.S. Application Data
(60) Provisional application No. 63/246,304, filed on Sep. 20, 2021.

(51) Int. Cl. A61L 2/10; A61L 2/26

(52) U.S. Cl. CPC A61L 2/10; A61L 2/26; A61L 2202/11; A61L 2202/122; A61L 2202/14; A61L 2202/121

(57) ABSTRACT

Disclosed solutions include an apparatus for sanitizing devices. The apparatus includes a flexible pad and an enclosure. The flexible pad includes light sources dispersed throughout the flexible pad and configured to emit ultraviolet light. The flexible pad is configured to diffuse the emitted light. The enclosure includes an opening through which the flexible pad can be extended and retracted.

### CROSS-REFERENCES TO RELATED APPLICATIONS

This application claims the benefit of U.S. Provisional Application No. 63/246,304 filed Sep. 20, 2001, the contents of which is hereby incorporated by reference in their entirety.

### TECHNICAL FIELD

This invention relates generally to an apparatus for sanitizing objects and more specifically to a diffusing pad that emits ultraviolet light to clean computing devices such as keyboards.

### BACKGROUND

Many everyday objects are exposed to pathogens such as viruses, but these everyday objects are rarely sanitized. For example, electronic devices, ever prevalent on in today's world, are rarely cleaned. Moreover, cleaning these devices can be difficult. For example, applying cleaning fluids to electronic devices can damage sensitive electronics. Hence, new solutions are needed.

### SUMMARY

Certain embodiments include an apparatus for sanitizing devices. The apparatus includes a flexible pad and an enclosure. The flexible pad includes light sources dispersed throughout the flexible pad and configured to emit ultraviolet light. The flexible pad is configured to diffuse the emitted light. The enclosure includes an opening through which the flexible pad can be extended and retracted.

Certain methods include extending a flexible pad of a sanitizing apparatus over a surface of a computing device and cleaning a surface of the computing device by laying the apparatus on the surface to be cleaned. The apparatus may emit ultraviolet light, start a timer, and upon expiration of the timer, stop emitting ultraviolet light.

Additional methods include extending the flexible pad over a keyboard of a clamshell laptop and activating the apparatus to emit ultraviolet light onto the keyboard. The method may continue by closing the clamshell laptop causing the apparatus to be between a screen of the clamshell laptop and the keyboard. The method may further include starting a timer and upon expiration of the timer, causing the apparatus to stop emitting ultraviolet light.

These illustrative examples are mentioned not to limit or define the disclosure, but to provide examples to aid understanding thereof. Additional examples and further description are provided in the Detailed Description.

### BRIEF DESCRIPTION OF THE FIGURES

These and other features, embodiments, and advantages of the present disclosure are better understood when the following Detailed Description is read with reference to the accompanying drawings, where:

FIG. 1 depicts a side view of an exemplary sanitizing apparatus, in accordance with an embodiment of the present invention.

FIG. 2 depicts a front view of an exemplary sanitizing apparatus, in accordance with an embodiment of the present invention.

FIG. 3 depicts a side view of an exemplary sanitizing apparatus, in accordance with an embodiment of the present invention.

FIG. 4 depicts an exemplary use case of a sanitizing apparatus, in accordance with an embodiment of the present invention.

FIG. 5 depicts a side view of an exemplary sanitizing apparatus, in accordance with an embodiment of the present invention.

FIG. 6 depicts an exploded view of an enclosure of a sanitizing apparatus, in accordance with an embodiment of the present invention.

FIG. 7 depicts a lower case of an enclosure of a sanitizing apparatus, in accordance with an embodiment of the present invention.

FIG. 8 depicts an exemplary printed circuit board for a controller system of a sanitizing apparatus, in accordance with an embodiment of the present invention.

FIG. 9 illustrates an exemplary a computing device, according to certain embodiments of the present disclosure.

### DETAILED DESCRIPTION

Embodiments of the present invention relate to an apparatus for sanitizing objects such as computing devices. Disclosed techniques use an electronically-controlled diffusing pad (or surface) that emits ultraviolet light in a substantially uniform fashion, thereby sanitizing objects placed beneath. Accordingly, disclosed solutions use ultraviolet light at wavelengths suitable to kill microbes such as bacteria and viruses. Examples of suitable ranges of wavelengths of light include ultraviolet light at 100-400 nanometers (nm), including ultraviolet-C UVC (200-280 nm) and far-UVC (207-222 nm). In an example, UV-C light at wavelength of 222 nm is used. Disclosed solutions employ a convenient, retractable diffusing pad that can be adjusted to accommodate a large surface such as a laptop keyboard, while maintaining a small footprint while retracted.

In an exemplary embodiment, a sanitizing apparatus includes a diffusing pad that can be extended over a keyboard. The diffusing pad includes multiple light sources, such as Light Emitting Diodes (LEDs), that are configured to emit ultraviolet light. The diffusing pad is formed of a material suitable for diffusing the light emitted from the LEDs to be more substantially uniform in distribution. The sanitizing apparatus can include an enclosure, which can include a power source, an electronic controller or processor, and one or more controls. The apparatus can be configured to emit light for a specific amount of time. In example use case, to clean a surface, the diffusing pad of the apparatus is placed over one or more objects. The user engages a power button located on the enclosure. A controller housed inside the enclosure then activates the LEDs to emit ultraviolet light for a predetermined period of time. The germicidal capabilities of the ultraviolet light then clean the surface. After the timer expires, the controller disables power to the LEDs. The diffusing pad turns off, thereby ending the cleaning cycle.

During experimentation, an exemplary sanitizing apparatus was configured to expose a petri dish of bacteria to ultraviolet light in various configurations. The different configurations included modifying a distance from the petri dish to the light source, e.g. from several centimeters to below one millimeter and modifying a time period, from minutes to hours, during which the bacteria was exposed to ultraviolet light. Observations included a diminished quantity of bacteria. Experimentation determined that a specific radius of each individual ultraviolet light source was identified, at which the light source had germicidal capabilities. In one experiment, it was identified that germicidal capabilities had a radii limit of 3.5 cm, and beyond that limit, no discernible bacteria killing effect was observed. In another example, a configuration with less than one minute of light exposure was able to reduce the quantity of bacteria. This configurations can be adjusted via distance, power, wavelength, etc.

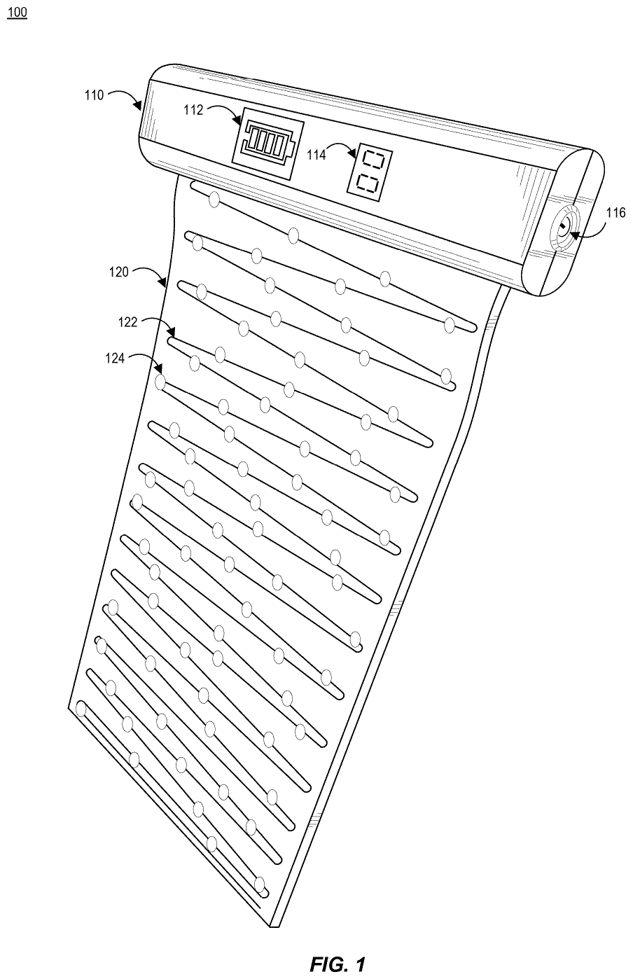

Turning now to the Figures, FIG. 1 depicts a side view of an exemplary sanitizing apparatus 100 for cleaning objects, in accordance with an embodiment of the present invention. Apparatus 100 includes enclosure 110 and diffusing pad 120. In the example depicted, diffusing pad 120 is configured to provide ultraviolet light (e.g., UV-C) to sanitize one or more devices.

Enclosure 110 includes battery indicator 112, display 114, and power button 116. Battery indicator 112 can be configured to indicate a remaining capacity of an internal battery, if installed. Display 114 can be configured to display various feedback information such as a remaining time left until a device is sufficiently sanitized, or a time elapsed during sanitation. While depicted as having two digits, display 114 can have any number of digits and/or display capabilities. Power button 116 is configured to turn power to the apparatus 100 on or off. Examples of power button 116 include a push-button switch, rocker switch, and so forth.

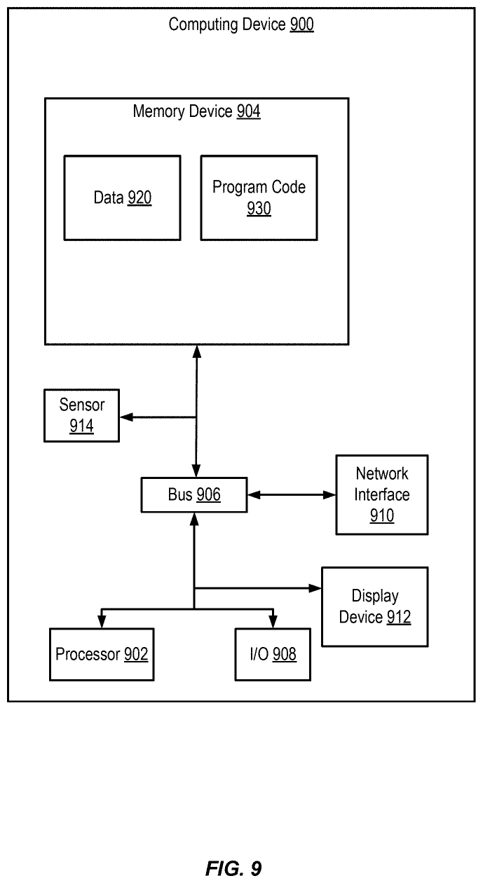

Additionally or alternatively, enclosure 110 can house additional controls and/or electronic devices. For example, enclosure 110 can include one or more processors or controllers, a suitable example of which is explained further with respect to computing device 900 in FIG. 9. Suitable processors include ARM-based microcontrollers such as Arduino. Such a processor can perform various controller functions such as enabling or disabling the light sources, adjusting light settings, setting timers, controlling charging of internal batteries, and so forth. In some embodiments, the processors can control a motor that can extend and/or retract diffusing pad 120. In some embodiments, a control system to control the light sources can be made without a dedicated programmable controller or processor but instead with a custom circuit.

In some embodiments, enclosure 110 is not present and the sanitizing apparatus 100 includes only the diffusing pad 120 and a power source. For instance, in one embodiment, the diffusing pad 120 could have a port (e.g., USB) to which power is connected but no internal battery or additional controls.

The processor can be configured to control and update battery indicator 112 and/or display 114. For example, the processor can periodically monitor the remaining battery charge and update battery indicator 112 accordingly. In another example, the processor can execute a timer and periodically (e.g., every second) update display 114 with a remaining time.

The processor can also receive measurements from an inertial sensor such as a gyroscope and/or accelerometer. In some cases, the inertial measurement sensor allows for an implementation of an automatic shutoff system to prevent excessive exposure to ultraviolet light. For example, the inertial measurement sensor can be used to detect whether enclosure 110 and/or diffusing pad 120 are rotated or incorrectly oriented.

For example, a gyroscope installed internally within enclosure 110 or diffusing pad 120 can determine an angle or orientation of enclosure 110. The processor can receive data from the gyroscope, translate the data into a suitable form such as an angle measurement, and determine if the angle is beyond a threshold. When the angle is beyond the threshold, then the processor determines that enclosure 110 has been rotated such that emitted light may be emitted upwards, i.e., away from the desired object to be sanitized. As exposure to UV light may be in some cases harmful, the use of the gyroscope may protect users from accidental exposure to the UV light.

In some embodiments, a tilt switch can be used to implement the automatic shutoff system. The tilt switch can be mounted within enclosure 110 or diffusing pad 120. When the tilt switch is oriented past a specific threshold (e.g., degree), the tilt switch can break a circuit and stop current flow to the light sources 124. In this manner, a processor and gyroscope are not needed.

In some embodiments, if apparatus 100 suddenly impacts an object such as a table or a floor, then the processor can disable the light sources 124. In other embodiments, one or more tilt sensors and/or light sensors can be used to determine whether the processor disables the light source.

In some embodiments, a timer can be used to guide a user on how long a device must be exposed to light to be sanitized. Examples of suitable sanitation times include five minutes and fifteen minutes. Longer or shorter times are possible. For instance, if a sanitization takes 1 minute, then once a light source is activated, then the timer can count down from one minute. In some cases, an audible or visual alarm is activated when the timer is complete. The timer allows sanitizing apparatus 100 to passively sanitize, so there is no action required from the user after turning it on. In addition, the automatic shutoff controlled by the timer is also a safety precaution, preventing excess UV-C exposure as well as prolonging the lifespan of the device. In some embodiments, the devices shuts off automatically after 15 minutes. In some embodiments, a user may have an option to select between different cleaning times and/or light intensities. For example, a user may select from a quick and convenient five-minute clean and a long, thorough fifteen-minute clean. Additionally or alternatively, the user can set the sanitization time manually to fine-tune the cleaning cycle and may stop an ongoing cycle at any time via the power button or other controls.

Enclosure 110 may be battery powered or powered by an external source. For example, a battery can be mounted inside enclosure 110. In some embodiments, this battery is rechargeable. For example, enclosure 110 can include a charging port to which an external power supply can be provided to charge and/or power the light sources 124. Examples of suitable charging ports include Universal Serial Bus (USB) ports, including USB type A and USB type C. In some embodiments, enclosure 110 may lack a battery and may be powered through USB or other power sources.

In some embodiments, enclosure 110 can also use an existing USB port or have one or more additional USB ports for powering and data. For example, a USB port can facilitate data transfer to or from other devices such as a phone or flash drive. The USB port can be connected to the charger port of enclosure 110 to function as a data transferable USB hub. In another example, sanitizing apparatus 100 is positioned on top of a keyboard of a computing device and simultaneously draws power from the computing device's USB port.

Enclosure 110 can be formed of one or more materials such as metal, plastic, or wood. Enclosure 110 can be molded from a custom mold or formed of plastic generated by a three-dimensional (3D) printer.

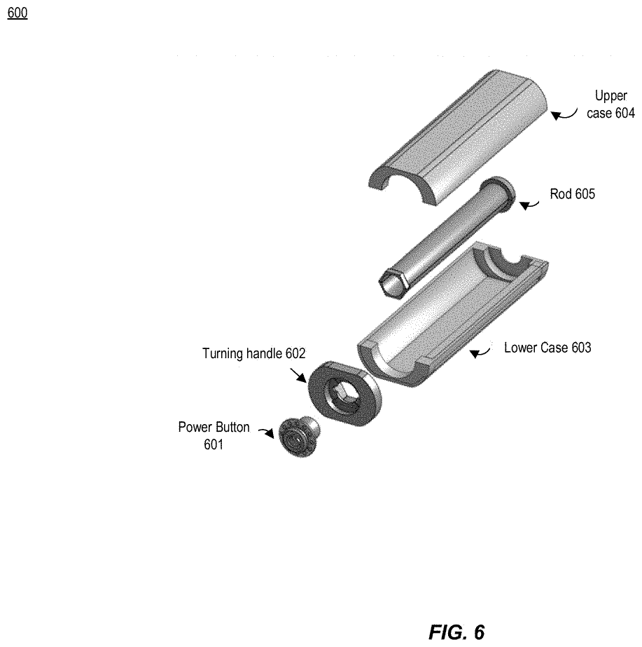

In some embodiments, diffusing pad 120 is retractable manually, e.g., by turning a knob (not shown). The knob can be attached to a rod around which the pad is wrapped. Enclosure 110 can receive the diffusing pad 120 via an opening and roll the diffusing pad 120 around a rod for storage. To release the diffusing pad 120, a user can pull on an end of the diffusing pad 120 to extend the diffusing pad from enclosure 110. In some embodiments, enclosure 110 may be opened with a hinge for access to internal electronics and/or troubleshooting. An exemplary internal mechanism is described with respect to FIG. 6.

In yet other embodiments, diffusing pad 120 can be rolled up adjacent to or around enclosure 110. In other embodiments, diffusing pad 120 is not retractable and is affixed to enclosure 110. In another embodiment, diffusing pad 120 is automatically retractable upon demand via one or more motors present in enclosure 110.

In yet other embodiments, diffusing pad 120 is retractable via a spring-loaded mechanism. In this example, a spring gains tension when diffusing pad 120 is manually pulled out and the device is locked in place when the pad is fully extended (possibly by ‘hooking’ over a computing device). When the diffusing pad 120 is to be retracted, the lock is disengaged via a button or an automatic control and the tensed spring pulls the pad back in. Examples of suitable springs include rotary or strip springs.

Diffusing pad 120 includes one or more light sources 124. The light sources 124 can be configured to emit ultraviolet light. Examples of light sources 124 include Light Emitting Diodes (LEDs). The light sources 124 are connected via wire 122. The light sources 124 can be arranged across diffusing pad 120 in various configurations. For instance, as depicted, light sources 124 are arranged roughly equidistant in a zig-zag pattern across diffusing pad 120. But any arrangement is possible. Other examples include a pixelated grid arrangement. In some cases, a density of light sources 124 can be varied, for example, to provide more light on particularly dirty areas of the object.

Diffusing pad 120 can be formed of a flexible substrate that is capable of curvature. Examples include silicone, rubber, latex, or flexible plastic. But other materials are possible. In an example, the diffusing pad 120 is formed of antistatic premium silicone that is 3 mm thick. In some embodiments, the diffusing pad is opaque or transparent. In some cases, diffusing pad 120 can be stretched past its original dimensions.

Diffusing pad 120 can be formed from a mold that is generated via a 3D printer. For example, a string or array (e.g., a grid) of LEDs can be placed into the mold and a liquid substance poured into the mold such that the LEDs are integrated into the diffusing pad.

In some embodiments, diffusing pad 120 can employ fiber optic cables or light diffusing acrylic to diffuse the light emitted from light sources 124. In this case, the light sources 124 can be located inside of enclosure 110, and diffusing pad 120 can be formed from diffusing fiber optic or acrylic materials. Light sources 124 can be focused on the diffusing material, which can spread light evenly across diffusing pad 120, without the need to embed lights directly in diffusing pad 120. Fiber optic cables can be placed across and within the diffusing pad 120. Other diffusing materials include rubber and plastic. In some cases, this approach may be more economically feasible as fluorescent light tubes are more available and fewer light sources are needed due to the diffusion.

In some embodiments, one side of diffusing pad 120 may have a light-blocking or reflecting layer, allowing the light to face only one direction, e.g., downward. In other embodiments, the diffusing pad does not have a reflective layer such that it can be inserted between a laptop keyboard and screen with the laptop clamshell closed such that both screen and keyboard can be sanitized. In yet other embodiments, some of light sources 124 point upwards and some point downwards, facilitating sanitization of two surfaces. In this manner, the upward-pointing and downward-pointing light sources can be separately activated or deactivated.

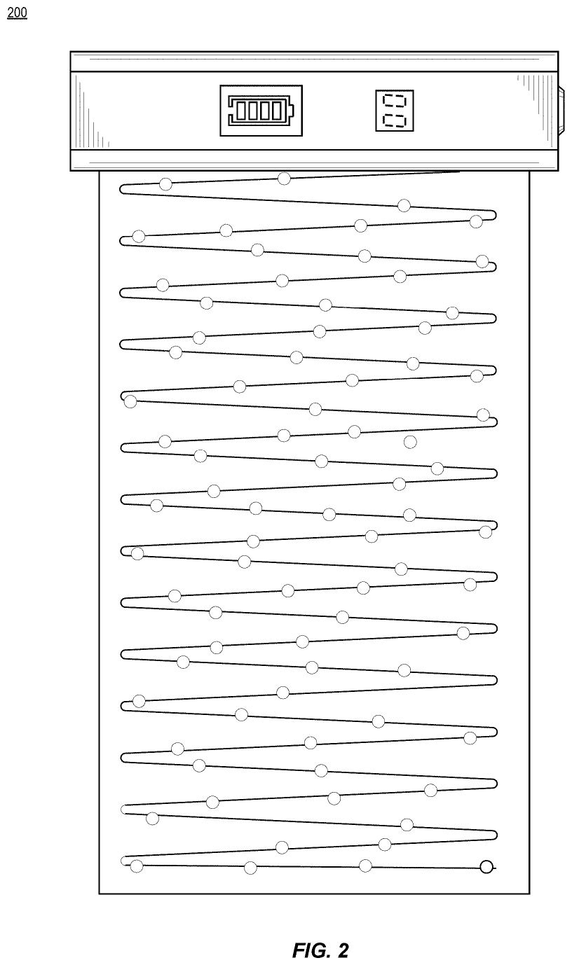

FIG. 2 depicts a front view of an exemplary sanitizing apparatus 200, in accordance with an embodiment of the present invention.

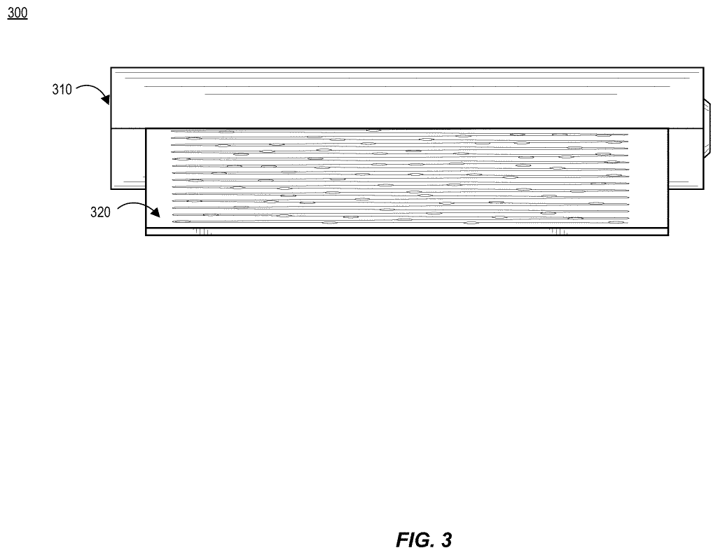

FIG. 3 depicts a side view of an exemplary a sanitizing apparatus 300, in accordance with an embodiment of the present invention. Sanitizing apparatus 300 includes enclosure 310 and pad 320. As can be seen, pad 320 is partially rolled up within sanitizing apparatus 300.

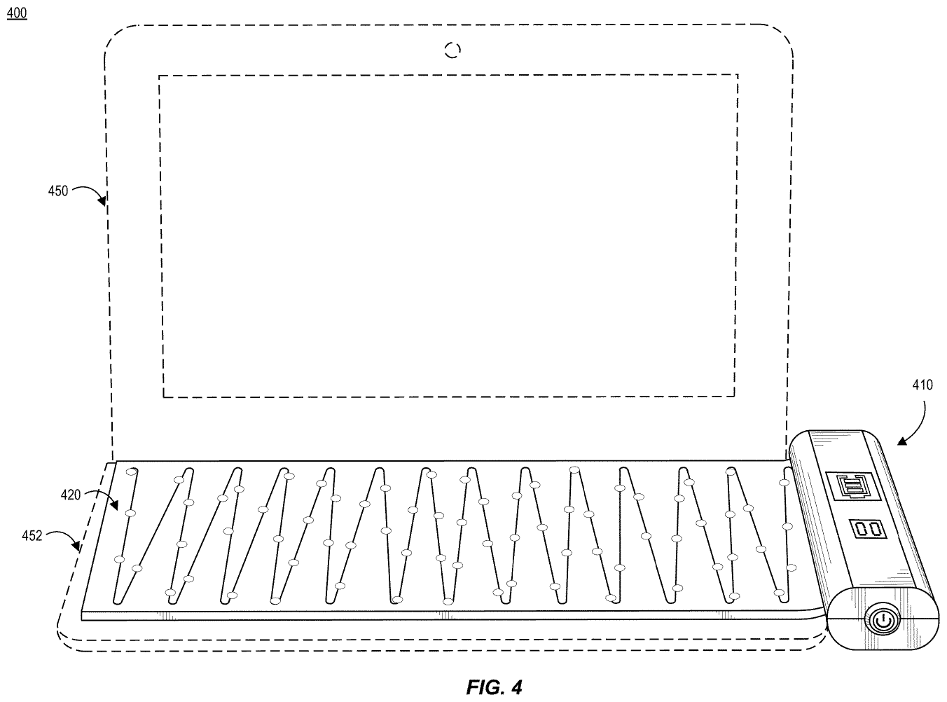

FIG. 4 depicts an exemplary use case of a sanitizing apparatus, in accordance with an embodiment of the present invention. FIG. 4 depicts use case 400, which shows enclosure 510, diffusing pad 420 and computing device 450. While as depicted, diffusing pad 420 is placed on top of keyboard 452, covering substantially all of keyboard 452, diffusing pad 420 can be retracted or expanded to accommodate keyboards of different sizes.

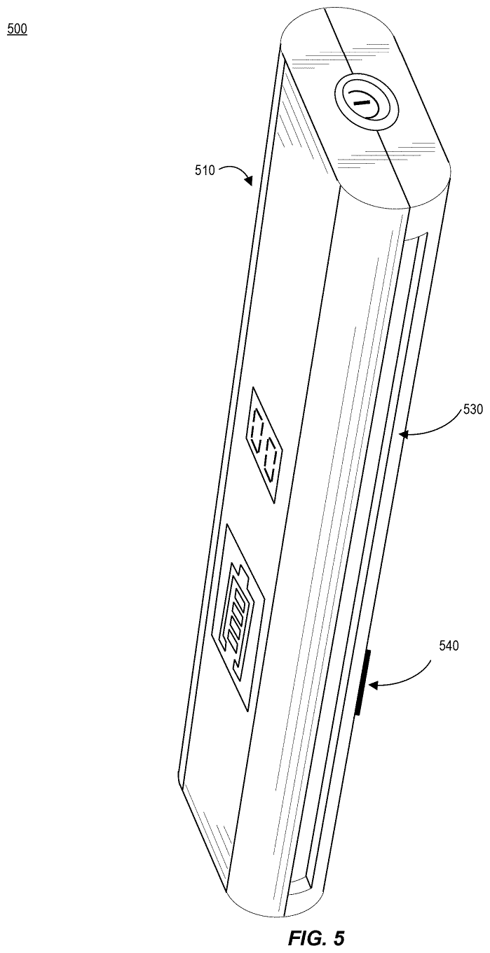

FIG. 5 depicts a side view of an exemplary sanitizing apparatus 500, in accordance with an embodiment of the present invention. Sanitizing apparatus 500 includes enclosure 510, which includes opening 530 and light sensor 540. The diffusing pad can be inserted or retracted through opening 530. Light sensor 540 is used to detect a presence of light below sanitizing apparatus 500.

FIG. 6 depicts an exploded view of an enclosure of a sanitizing apparatus, in accordance with an embodiment of the present invention. Enclosure 600 includes power button 601, turning handle 602, lower case 603, upper case 604, and rod 605. Power button 601 turns the apparatus on or off. Turning handle 602 is connected to rod 605. When turning handle 602 is turned, the rod 605 rotates to retract the pad either into the enclosure or expand the pad from the enclosure. Lower case 603 and upper case 604 can attach to each other to form the enclosure.

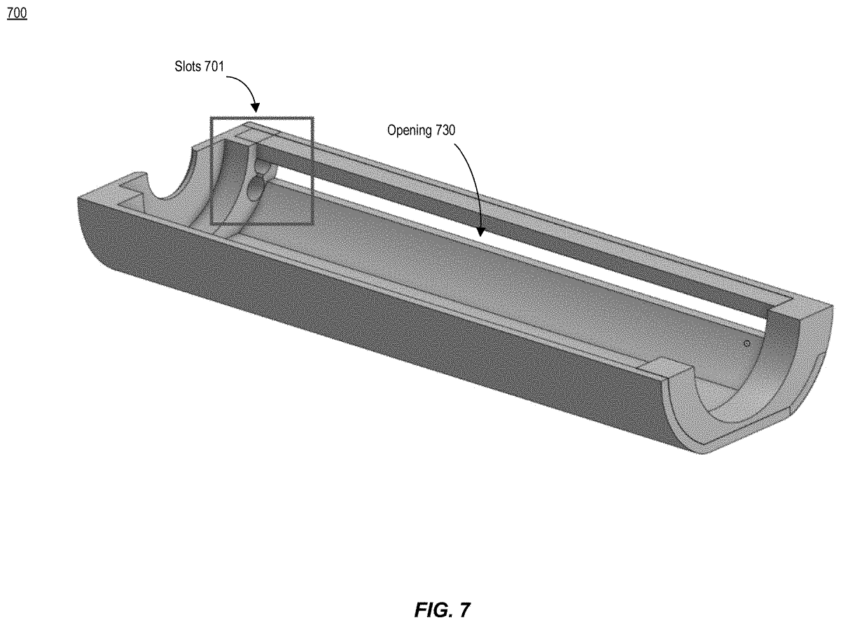

FIG. 7 depicts a lower case of an enclosure of a sanitizing apparatus, in accordance with an embodiment of the present invention. Enclosure 700 includes one or more slots 701, and opening 730. Slots 701 are designed to receive two rods or rollers that can rotate and allow the pad to roll out of the enclosure. For example, the diffusing pad can be inserted between the two rods or rollers such that the diffusing pad is held in place. The rods also reduce the friction applied on the pad.

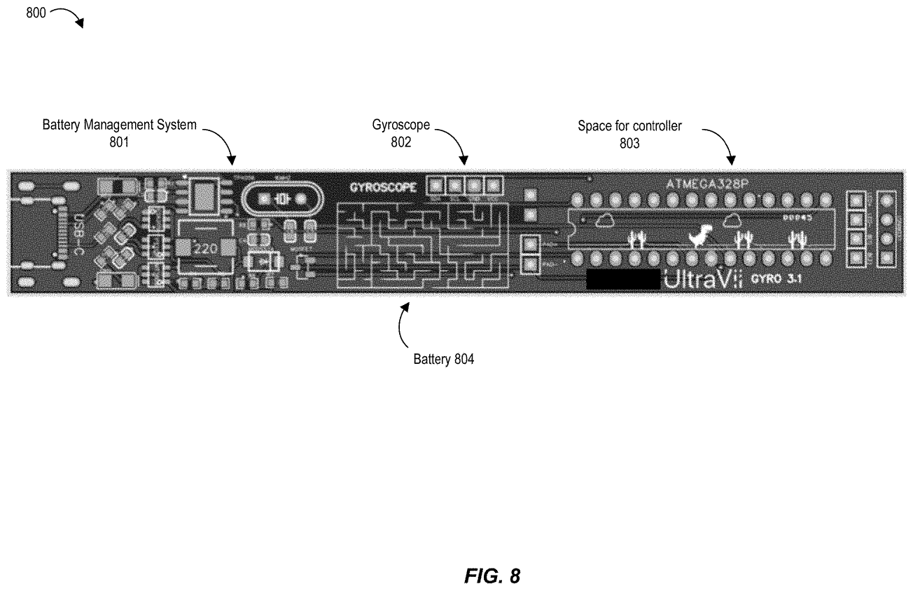

FIG. 8 depicts an exemplary printed circuit board 800 for a controller system of a sanitizing apparatus, in accordance with an embodiment of the present invention. Circuit board 800 includes spaces for various devices including battery management system 801, gyroscope 802, controller 803, and battery 804 (on the rear side). Battery management system 801 is a dedicated system for controlling the battery 804 including charging, if applicable. In some embodiments, these functions can be performed by controller 803. Examples of controller 803 include computing device 900, described with respect to FIG. 9.

FIG. 9 illustrates an exemplary a computing device, according to certain embodiments of the present disclosure. Any suitable computing device may be used for performing the operations described herein. The depicted example of a computing device 900 includes a processor 902 communicatively coupled to one or more memory devices 904. Computing device 900 can be used in a meter, collector, or any other device described herein. The processor 902 executes computer-executable program code 930 stored in a memory device 904, accesses data 920 stored in the memory device 904, or both. Examples of the processor 902 include a microprocessor, an application-specific integrated circuit (“ASIC”), a field-programmable gate array (“FPGA”), or any other suitable processing device. The processor 902 can include any number of processing devices or cores, including a single processing device. The functionality of the computing device may be implemented in hardware, software, firmware, or a combination thereof.

The memory device 904 includes any suitable non-transitory computer-readable medium for storing data, program code, or both. A computer-readable medium can include any electronic, optical, magnetic, or other storage device capable of providing a processor with computer-readable instructions or other program code. Non-limiting examples of a computer-readable medium include a flash memory, a ROM, a RAM, an ASIC, or any other medium from which a processing device can read instructions. The instructions may include processor-specific instructions generated by a compiler or an interpreter from code written in any suitable computer-programming language, including, for example, C, C++, C#, Visual Basic, Java, or scripting language.

The computing device 900 may also include a number of external or internal devices, such as input or output devices. For example, the computing device 900 is shown with one or more input/output (“I/O”) interfaces 908. An I/O interface 908 can receive input from input devices or provide output to output devices. One or more busses 906 are also included in the computing device 900. The bus 906 communicatively couples one or more components of a respective one of the computing device 900.

The computing device 900 executes program code 930 that configures the processor 902 to perform one or more of the operations described herein. For example, the program code 930 can cause the processor to perform the operations described in FIG. 3.

The computing device 900 also includes a network interface device 910. The network interface device 910 includes any device or group of devices suitable for establishing a wired or wireless data connection to one or more data networks. The network interface device 910 may be a wireless device and have an antenna. The computing device 900 can communicate with one or more other computing devices implementing the computing device or other functionality via a data network using the network interface device 910.

The computing device 900 can also include a display device 912. Display device 912 can be a LCD, LED, touch-screen or other device operable to display information about the computing device 900. Examples of display device 912 include battery indicator 112 and/or display 114. The computing device 900 can also include sensor 914. Examples of sensor 914 include inertial sensors such as accelerometers and gyroscopes.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation and does not preclude inclusion of such modifications, variations, and/or additions to the present subject matter as would be readily apparent to one of ordinary skill in the art.

While the present subject matter has been described in detail with respect to specific embodiments thereof, it will be appreciated that those skilled in the art, upon attaining an understanding of the foregoing, may readily produce alterations to, variations of, and equivalents to such embodiment. Accordingly, it should be understood that the present disclosure has been presented for purposes of example rather than limitation and does not preclude inclusion of such modifications, variations, and/or additions to the present subject matter as would be readily apparent to one of ordinary skill in the art.

What is claimed is:

1. A sanitizing apparatus comprising:
a flexible pad comprising a plurality of light sources, the plurality of light sources dispersed throughout the flexible pad and configured to emit ultraviolet light, wherein the flexible pad is configured to diffuse the emitted ultraviolet light; and
an enclosure comprising an opening that is configured to receive the flexible pad, wherein the flexible pad is retractable, wherein the sanitizing apparatus is configured to activate the plurality of light sources a period of time to sanitize an object that is adjacent to the flexible pad.

2. The sanitizing apparatus of claim 1, wherein the enclosure further comprises a first roller and a second roller through which the flexible pad is inserted.

3. The sanitizing apparatus of claim 1, wherein the enclosure comprises a rod that is attached to a first end of the flexible pad and a motor, wherein the motor is configured to rotate the roller, thereby retract the flexible pad into the enclosure.

4. The sanitizing apparatus of claim 1, wherein the flexible pad is formed of silicone.

5. The sanitizing apparatus of claim 1, wherein the flexible pad further comprises a reflective layer attached to or within a first side of the flexible pad, wherein the reflective layer is configured to reflect the emitted ultraviolet light.

6. The sanitizing apparatus of claim 1, wherein the flexible pad further is configured to emit ultraviolet light from a first surface oriented in a first direction and from a second surface oriented in a second direction.

7. The sanitizing apparatus of claim 1, wherein the enclosure comprises a controller configured to:
cause each of the plurality of light sources to be activated;
upon the activation, start a timer; and
upon an expiration of the timer, cause the plurality of light sources to be deactivated.

8. The sanitizing apparatus of claim 7, wherein the timer is adjustable.

9. The sanitizing apparatus of claim 7, wherein the enclosure includes a display, and wherein the controller is further configured to cause the display to output a remaining time of the timer.

10. The sanitizing apparatus of claim 1, wherein the light sources are configured to emit ultraviolet light at UV-C wavelengths.

11. The sanitizing apparatus of claim 1, wherein the enclosure comprises a controller and the sanitizing apparatus includes an inertial sensor, wherein the controller is configured to:
receive, from the inertial sensor, one or more inertial measurements;
determine, from the one or more inertial measurements, that the sanitizing apparatus is rotated beyond a threshold; and
responsive to determining, that the sanitizing apparatus is rotated a threshold, cause the plurality of light sources to be deactivated.

12. A method of using the sanitizing apparatus of claim 1, the method comprising:
extending the flexible pad over a surface of a computing device;
activating the sanitizing apparatus by causing the sanitizing apparatus to emit ultraviolet light; and
upon an expiration of a timer, causing the sanitizing apparatus to stop emitting ultraviolet light.

13. The method of claim 12, wherein the apparatus cleans the surface by emitting ultraviolet light at UV-C wavelengths.

14. A method of using the sanitizing apparatus of claim 1, the method comprising:
extending the flexible pad between a keyboard of a computing device and a screen of the computing device;
closing a clamshell of the computing device;
activating the sanitizing apparatus by causing the apparatus to emit ultraviolet light; and
upon an expiration of a timer, causing the sanitizing apparatus to stop emitting ultraviolet light.

15. A sanitizing apparatus comprising:
a flexible pad comprising:
a plurality of light sources dispersed throughout the flexible pad and configured to emit ultraviolet light,
a reflective layer positioned on a first surface of the flexible pad, wherein the flexible pad is configured to diffuse the emitted light and the reflective layer is configured to reflect the emitted light towards a second surface of the reflective pad;
an enclosure comprising:
an opening that is configured to receive the flexible pad, wherein the flexible pad is retractable, and
a roller that is attached to a first end of the flexible pad and is configured to retract and store the flexible pad; and
a processor configured to:
cause each of the plurality of light sources to be activated,
upon the activation, start a timer, and
upon an expiration of the timer, cause the plurality of light sources to be deactivated, wherein the activation of the plurality of light sources during the timed period causes an object that is adjacent to the second surface to be sanitized.

16. The sanitizing apparatus of claim 15, wherein each of the plurality of light sources is configured to emit light comprising a wavelength of 222 nanometers.

17. The sanitizing apparatus of claim 15, wherein the enclosure further comprises an inertial sensor, and wherein the processor is further configured to:
receive, from the inertial sensor, one or more inertial measurements;
determine, from the one or more inertial measurements, that the apparatus is rotated beyond a threshold; and
responsive to determining, that the apparatus is rotated a threshold, cause the plurality of light sources to be deactivated.
